(19) United States
(12) Patent Application Publication
(10) Pub. No.: US 2024/0164639 A1
(43) Pub. Date: May 23, 2024

(54) HARDNESS CALCULATION DEVICE, HARDNESS MEASUREMENT DEVICE, AND HARDNESS CALCULATION METHOD

(71) Applicant: TOHOKU UNIVERSITY, Sendai-shi, Miyagi (JP)

(72) Inventors: Akihiko YAMAGUCHI, Sendai-shi (JP); Toru NAKAZAWA, Sendai-shi (JP); Yasuhisa NEMOTO, Sendai-shi (JP)

(21) Appl. No.: 18/551,979

(22) PCT Filed: Mar. 29, 2021
(86) PCT No.: PCT/JP2021/013213

§ 371 Date: Sep. 22, 2023

(51) Int. Cl. A61B 3/16; A61B 3/00

(52) U.S. Cl. CPC A61B 3/16; A61B 3/0025; A61B 2562/0247; A61B 2562/0257

(57) ABSTRACT

A hardness calculation device includes a distance measurement information acquisition unit configured to acquire distance measurement information indicating a distance to a reference position that is a part of a body of an animal, a pressure information acquisition unit configured to acquire pressure information indicating a pressure when a contact section is pressed against a target position that is a part of the body of the animal, the target position being a position different from the reference position, a calculation unit configured to calculate hardness information that is information about a hardness of a tissue existing in the body of the animal at the target position based on the distance measurement information acquired by the distance measurement information acquisition unit and the pressure information acquired by the pressure information acquisition unit, and an output unit configured to output the hardness information calculated by the calculation unit.

### TECHNICAL FIELD

The present invention relates to a hardness calculation device, a hardness measurement device, and a hardness calculation method.

### BACKGROUND ART

Conventionally, when a patient suspected of having a disease is diagnosed by a doctor, the patient goes to see a doctor and the doctor makes a diagnosis by palpating an affected area of the patient. As an attempt to obtain a similar diagnostic result without relying on a skilled doctor, there has been a technique related to a palpation device that measures an elastic modulus of an affected area (for example, see Patent Document 1).

### CITATION LIST

### Patent Literature

Patent Document 1: JP 10-211172 A

### SUMMARY OF INVENTION

### Technical Problem

With such a palpation device, a doctor may be able to quantitatively measure the elastic modulus by wearing the palpation device. However, depending on an affected area or a disease, it is not sufficient to quantitatively measure the elastic modulus, and periodic measurement is required in some cases. In such a case, the patient has to regularly go to see a doctor, and there is a problem that the patient is burdened in order to be palpated by the doctor.

The present invention has been made in view of such circumstances, and an object thereof is to provide a hardness calculation device, a hardness measurement device, and a hardness calculation method capable of easily calculating the hardness of a part of a body.

### Solution to Problem

According to an aspect of the present invention, there is provided a hardness calculation device including a distance measurement information acquisition unit configured to acquire distance measurement information indicating a distance to a reference position that is a part of a body of an animal, a pressure information acquisition unit configured to acquire pressure information indicating a pressure when a contact section is pressed against a target position that is a part of the body of the animal, the target position being a position different from the reference position, a calculation unit configured to calculate hardness information that is information about a hardness of a tissue existing in the body of the animal at the target position based on the distance measurement information acquired by the distance measurement information acquisition unit and the pressure information acquired by the pressure information acquisition unit, and an output unit configured to output the hardness information calculated by the calculation unit.

Further, in the hardness calculation device according to an aspect of the present invention, the distance measurement information acquisition unit acquires a plurality of pieces of the distance measurement information acquired at different moments from each other, the pressure information acquisition unit acquires a plurality of pieces of the pressure information acquired at moments corresponding to the moments at which the plurality of pieces of the distance measurement information are acquired, and the calculation unit calculates the hardness information based on the acquired plurality of pieces of the distance measurement information and the acquired plurality of pieces of the pressure information.

Further, in the hardness calculation device according to an aspect of the present invention, the calculation unit calculates a first hardness when a distance to the reference position is a first distance and a second hardness when a distance to the reference position is a second distance, the second hardness being different from the first hardness.

Further, in the hardness calculation device according to an aspect of the present invention, the part of the body of the animal is an eyelid of a human, the first hardness is a hardness of the eyelid, and the second hardness is a hardness of an eyeball.

Further, a hardness measurement device according to an aspect of the present invention includes the above-described hardness calculation device, a distance measurement sensor configured to measure a distance to the reference position, the distance measurement sensor being configured to output the measured distance to the distance measurement information acquisition unit as the distance measurement information, and a pressure sensor configured to measure a pressure when the contact section is pressed against the target position, the pressure sensor being configured to output the measured pressure to the pressure information acquisition unit as the pressure information.

Further, in the hardness measurement device according to an aspect of the present invention, the pressure sensor includes a deformation section including a contact surface including the contact section, the deformation section being configured to deform in response to the pressure when the contact surface comes into contact with the target position, a marker provided on a back side of the contact surface, and an imaging unit configured to capture an image of the marker from the back side of the contact surface.

Further, in the hardness measurement device according to an aspect of the present invention, the deformation section is made of a transparent material, the imaging unit captures an image of both the marker and an object existing on a contact surface side of the deformation section, and the output unit outputs both the hardness information and the captured image.

Further, in the hardness measurement device according to an aspect of the present invention, the distance measurement sensor measures the distance to the reference position without contact.

Further, the hardness measurement device according to an aspect of the present invention further includes a posture sensor configured to detect at least an inclination, and the output unit outputs both the hardness information and information indicating the inclination detected when the hardness information is calculated.

Further, in the hardness measurement device according to an aspect of the present invention, the calculation unit further includes a correction unit configured to correct the hardness indicated by the calculated hardness information according to the information indicating the inclination.

Further, the hardness measurement device according to an aspect of the present invention further includes a diagnostic information acquisition unit configured to acquire diagnostic information that is information to be acquired according to a result of the hardness information output by the output unit, and a treatment device configured to perform a treatment included in the diagnostic information.

Further, according to an aspect of the present invention, there is provided a hardness calculation method including acquiring distance measurement information indicating a distance to a reference position that is a part of a body of an animal, contacting a target position that is a part of the body of the animal, the target position being a position different from the reference position, acquiring pressure information indicating a pressure applied when pressing against the target position by the contacting of the target position, calculating hardness information that is information about a hardness of a tissue existing in the body of the animal at the target position based on the distance measurement information acquired by the acquiring distance measurement information and the pressure information acquired by the acquiring of pressure information, and outputting the hardness information calculated by the calculating of hardness information.

Further, according to an aspect of the present invention, there is provided a hardness calculation device including a distance measurement information acquisition unit configured to acquire distance measurement information indicating a distance to a reference position of an inspection target object, a pressure information acquisition unit configured to acquire pressure information indicating a pressure when a contact section is pressed against a target position that is a part of the inspection target object, the target position being a position different from the reference position, a calculation unit configured to calculate hardness information that is information about a hardness of a tissue existing in a body of the animal at the target position based on the distance measurement information acquired by the distance measurement information acquisition unit and the pressure information acquired by the pressure information acquisition unit, and an output unit configured to output the hardness information calculated by the calculation unit.

### Advantageous Effects of Invention

According to the present invention, it is possible to provide the hardness calculation device, the hardness measurement device, and the hardness calculation method capable of easily calculating the hardness of a part of a body.

### BRIEF DESCRIPTION OF DRAWINGS

FIG. 1 is a diagram illustrating an outline of a hardness measurement device according to a first embodiment.

FIG. 2 is a diagram illustrating an example of a functional configuration of the hardness measurement device according to the first embodiment.

FIG. 3 is a diagram illustrating an example of a functional configuration of a hardness calculation device according to the first embodiment.

FIG. 4 is a diagram for explaining a relationship between a push-in amount and a force of the hardness calculation device according to the first embodiment.

FIG. 5 is a flowchart illustrating a series of operations of a hardness calculation method according to the first embodiment.

FIG. 6 is a diagram illustrating an example of a functional configuration of a tactile sensor according to a second embodiment.

FIG. 7 is a diagram illustrating an example of a functional configuration of a hardness measurement device according to the second embodiment.

FIG. 8 is a diagram for explaining a marker included in the tactile sensor according to the second embodiment.

FIG. 9 is a diagram for explaining deformation of a marker when a contact section included in the tactile sensor according to the second embodiment comes into contact with an eyelid.

FIG. 10 is a diagram for explaining a device used for verifying a concept of the hardness measurement device according to the second embodiment.

FIG. 11 is a diagram for explaining a method used for verifying the concept of the hardness measurement device according to the second embodiment.

FIG. 12 is a diagram for explaining an example of measurement results measured by the concept verification of the hardness measurement device according to the second embodiment.

FIG. 13 is a diagram illustrating an example of a functional configuration of a hardness calculation device according to a third embodiment.

FIG. 14 is a diagram illustrating an example of a functional configuration of a hardness calculation device according to a fourth embodiment.

FIG. 15 is a diagram illustrating an example of a functional configuration of a hardness calculation device according to a fifth embodiment.

FIG. 16 is a diagram illustrating an example of a diagnostic system according to a sixth embodiment.

FIG. 17 is a diagram illustrating an example of a functional configuration of a hardness calculation device according to the sixth embodiment.

### DESCRIPTION OF EMBODIMENTS

### First Embodiment

Hereinafter, embodiments of the present invention will be described with reference to the accompanying drawings.

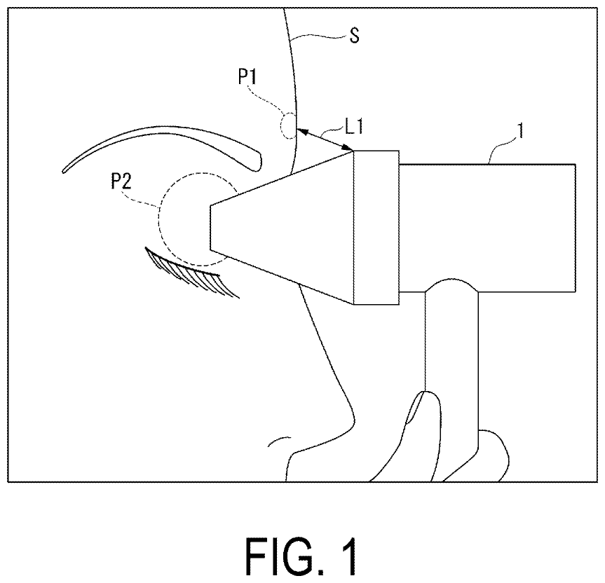

FIG. 1 is a diagram illustrating an outline of a hardness measurement device according to a first embodiment. An outline of a hardness measurement device 1 according to the first embodiment will be described with reference to the drawing. The hardness measurement device 1 measures the hardness of a part of the body of an animal. In the present embodiment, examples of the part of the body of the animal widely include the hardness of skin and tissues in the body covered by skin. In the following description, an example in which the part of the body of the animal is an eyelid or an eyeball of a human will be described.

Note that in the following description, components having the same functions are denoted by the same reference signs, and descriptions thereof may be omitted.

FIG. 1 illustrates an example in which a subject S measures an intraocular pressure of the subject S by using the hardness measurement device 1. The subject S operates the hardness measurement device 1 by himself/herself to measure his/her intraocular pressure. That is, in the present embodiment, the subject S himself/herself measures an affected area without the intervention of a doctor.

To be more specific, the subject S presses a probe portion of the hardness measurement device 1 against a position (target position) P2 to be measured.

Here, when the subject S presses the hardness measurement device 1 against the position P2, the pressing may be performed by using power generated from a drive source such as a motor or a pump (not illustrated). Further, an amount by which the subject S presses the hardness measurement device 1 against the position P2 may be an amount sufficient to measure an intraocular pressure.

The hardness measurement device 1 measures a pressure when the probe is pressed. Further, the hardness measurement device 1 measures a distance L1 to a position P1 different from the position P2. The hardness measurement device 1 calculates a hardness at the position P2 from a relationship between the measured pressure and the distance L1.

Here, the hardness at the position P2 may be the hardness of a skin surface (for example, an eyelid) at the position P2, or may be the hardness of a tissue (for example, an eyeball) existing inside the skin at the position P2.

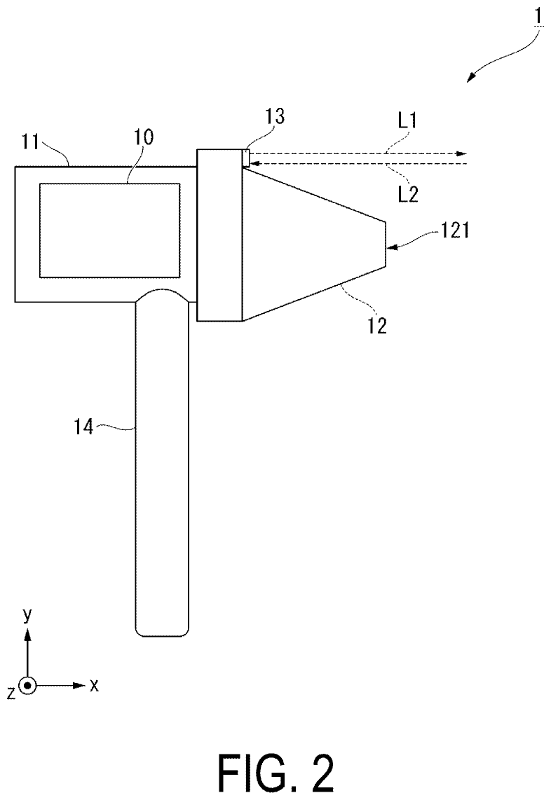

FIG. 2 is a diagram illustrating an example of a functional configuration of the hardness measurement device according to the first embodiment. An example of a functional configuration of the hardness measurement device 1 will be described with reference to the same drawing.

The hardness measurement device 1 includes a housing section 11, a pressure sensor 12, a distance measurement sensor 13, and a hold section 14.

The pressure sensor 12 includes a contact surface (contact section) 121 and measures a pressure (pressing force) when the contact surface 121 is pressed against a target position to be measured. The pressure sensor 12 may be, for example, a film-type pressure sensor that measures a pressure from a contact area of an electrode, a gauge sensor that measures a pressure from a change in the gauge resistance of a diaphragm surface, or the like.

The distance measurement sensor 13 measures a distance to a reference position. The distance measurement sensor 13 is, for example, a non-contact type sensor, and measures the distance to the reference position by detecting a light beam L2 that is a reflection light after a light beam L1 emitted from a light emitting unit (not illustrated) is reflected by a target object. When the distance measurement sensor 13 is a non-contact type sensor, the distance measurement sensor 13 may be, for example, an ultrasonic sensor or an infrared sensor.

Note that the distance measurement sensor 13 is not limited to a non-contact type sensor as an example, but may be a contact type sensor including a linear encoder or the like. A contact-type sensor may be used to measure distances with high accuracy.

Note that the distance measurement sensor 13 may measure distances at a plurality of points. The distance measurement sensor 13 may be able to more accurately measure a distance by averaging pieces of distance information corresponding to a plurality of points, for example.

The housing section 11 houses the hardness calculation device 10 therein.

The hardness calculation device 10 calculates a hardness based on the pressure measured by the pressure sensor 12 and the distance measured by the distance measurement sensor 13.

The hold section 14 is held by a user who uses the hardness calculation device 10. The user may be a person whose hardness of a part of the body is measured by the hardness calculation device 10, may be a caregiver or a helper when the user is a person requiring nursing care or the like and is not able to perform measurement, or may be an assistant who assists in measurement.

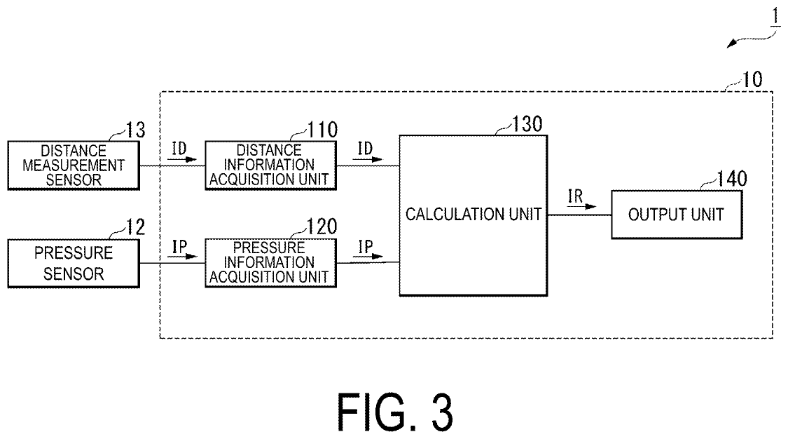

FIG. 3 is a diagram illustrating an example of a functional configuration of the hardness calculation device according to the first embodiment. An example of a functional configuration of the hardness calculation device 10 will be described with reference to the same drawing.

The hardness calculation device 10 includes a distance information acquisition unit (distance measurement information acquisition unit) 110, a pressure information acquisition unit 120, a calculation unit 130, and an output unit 140.

The distance information acquisition unit 110 acquires distance measurement information ID measured by the distance measurement sensor 13. The distance measurement information ID is information indicating a distance to a reference position that is a part of the body of an animal. The distance measurement sensor 13 measures the distance to the reference position and outputs the measured distance to the distance information acquisition unit 110 as the distance measurement information ID. That is, the distance information acquisition unit 110 acquires distance measurement information indicating the distance to the reference position that is the part of the body of the animal.

The pressure information acquisition unit 120 acquires pressure information IP from the pressure sensor 12. The pressure information IP is information indicating a pressure measured when the contact surface 121 of the probe is pressed against a target position that is a position different from the reference position. The pressure sensor 12 measures the pressure when the contact section is pressed against the target position, and outputs the measured pressure to the pressure information acquisition unit 120 as the pressure information IP. That is, the pressure information acquisition unit 120 acquires the pressure information IP indicating the pressure when the contact surface 121 is pressed against the target position that is the part of the body of the animal and that is the position different from the reference position.

The calculation unit 130 calculates hardness information IR based on the distance measurement information ID acquired by the distance information acquisition unit 110 and the pressure information IP acquired by the pressure information acquisition unit 120. The hardness information IR is information related to the hardness of a tissue existing in the body of the animal at the target position. That is, based on the distance measurement information ID and the pressure information IP, the calculation unit 130 calculates the hardness information IR that is information related to the hardness of the tissue existing in the body of the animal at the target position.

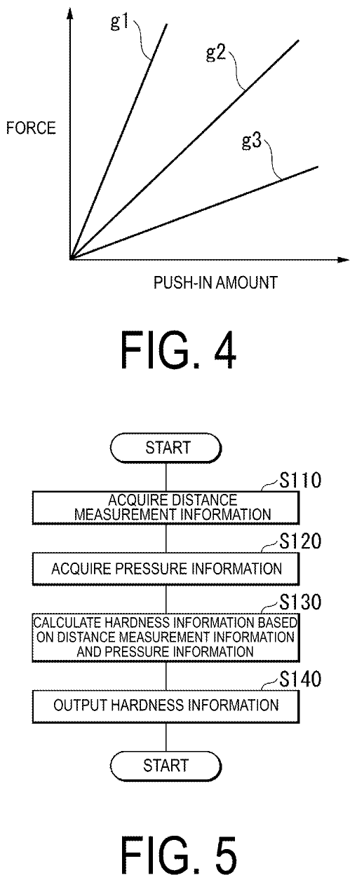

FIG. 4 is a diagram for explaining a relationship between a push-in amount and a force of the hardness calculation device according to the first embodiment. An example of the hardness information IR calculated by the calculation unit 130 will be described with reference to the same drawing. In the same drawing, the horizontal axis represents a “push-in amount” and the vertical axis represents a “force”, and a correspondence relationship between the push-in amount and the force is illustrated. The “push-in amount” indicates a distance by which the hardness measurement device 1 is pushed against a target position. The “push-in amount” is derived based on the distance measurement information ID. The “force” is a pressure at the target position. The “force” is derived based on the pressure information IP.

The calculation unit 130 stores the relationship between the “push-in amount” and the “force” in association with each other to calculate a graph as illustrated in FIG. 4, and calculates an inclination thereof as the hardness information IR. The hardness calculation device 10 continuously acquires the distance measurement information ID and the pressure information IP in order to acquire the correspondence relationship between the “push-in amount” and the “force”. That is, the distance information acquisition unit 110 acquires a plurality of pieces of the distance measurement information ID acquired at different moments from each other, the pressure information acquisition unit 120 acquires a plurality of pieces of the pressure information IP acquired at moments corresponding to the moments when the pieces of the distance measurement information ID are acquired, and the calculation unit 130 calculates the hardness information IR based on the acquired plurality of pieces of the distance measurement information ID and the acquired plurality of pieces of the pressure information IP.

In FIG. 4, three different measurement results are indicated by a line g1, a line g2 and a line g3. For example, the hardness indicated by the line g1 has an inclination sharper than an inclination of the hardness indicated by the line g2. That is, the hardness indicated by the line g1 is harder than the hardness indicated by the line g2. In addition, the hardness indicated by the line g3 has an inclination gentler than the inclination of the hardness indicated by the line g2. That is, the hardness indicated by the line g3 is softer than the hardness indicated by the line g2.

Referring back to FIG. 3, the output unit 140 outputs the hardness information IR calculated by the calculation unit 130. For example, the output unit 140 may include a communication unit (not illustrated) and may output the hardness information IR through a predetermined network such as the Internet or a Wi-Fi network.

Note that the hardness measurement device 1 may include a display unit (not illustrated), and the output unit 140 may output the hardness information IR to the display unit. The display unit may be, for example, a liquid crystal display, an organic electroluminescence (EL) display, or the like.

FIG. 5 is a flowchart illustrating a series of operations of a hardness calculation method according to the first embodiment. The series of operations of the hardness measurement device 1 will be described with reference to the same drawing.

(Step S110) The distance measurement sensor 13 measures a distance from a predetermined position in the hardness measurement device 1 to a reference position. The distance measurement sensor 13 outputs the measurement result as distance measurement information ID to the distance information acquisition unit 110. The distance information acquisition unit 110 acquires the distance measurement information ID from the distance measurement sensor 13.

(Step S120) The pressure sensor 12 measures a pressure when the contact surface 121 is pressed against a target position to be measured. The pressure sensor 12 outputs the measurement result as pressure information IP to the pressure information acquisition unit 120. The pressure information acquisition unit 120 acquires the pressure information IP from the pressure sensor 12.

(Step S130) The calculation unit 130 acquires the distance measurement information ID from the distance information acquisition unit 110, and acquires the pressure information IP from the pressure information acquisition unit 120. The calculation unit 130 calculates hardness information IR based on the acquired distance measurement information ID and the acquired pressure information IP.

(Step S140) The output unit 140 outputs the calculated hardness information IR.

### Summary of First Embodiment

According to the embodiment described above, the hardness calculation device 10 includes the distance information acquisition unit 110 to acquire distance measurement information ID, and includes the pressure information acquisition unit 120 to acquire pressure information IP. In addition, the hardness calculation device 10 includes the calculation unit 130 to calculate hardness information IR based on the distance measurement information ID and the pressure information IP, and includes the output unit 140 to output the hardness information IR. The distance measurement information ID and the pressure information IP are values obtained when a user presses the hardness measurement device 1 against a target position. Thus, according to the present embodiment, the hardness calculation device 10 can easily calculate the hardness of a part of the body without the patient being palpated by a doctor. Accordingly, the patient is saved the trouble of regularly going to see a doctor. In addition, since the patient can calculate the hardness of a part of the body by himself/herself at home, periodic measurement can be easily performed.

Additionally, according to the embodiment described above, the hardness calculation device 10 calculates the hardness information IR based on a plurality of pieces of distance measurement information ID acquired at different moments from each other and a plurality of pieces of pressure information IR acquired at moments corresponding to the moments at which the pieces of the distance measurement information ID are acquired. That is, the hardness calculation device 10 calculates the hardness information IR based on correspondence relationships between the “distances” and the “pressures” measured at a plurality of timings. Thus, according to the present embodiment, a hardness (inclination) corresponding to a push-in position can be calculated.

According to the embodiment described above, the hardness measurement device 1 includes the distance measurement sensor 13 to acquire distance measurement information ID, and includes the pressure sensor 12 to acquire pressure information IP. Thus, the hardness measurement device 1 does not require a large-sized configuration, and the device can be reduced in size. Accordingly, since a user can obtain the hardness measurement device 1 at a low price, the user can perform measurement by himself/herself without regularly going to see a doctor.

### Second Embodiment

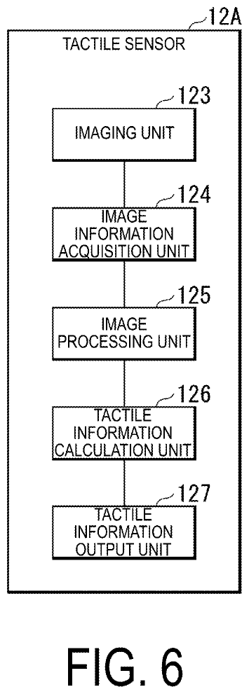

FIG. 6 is a diagram illustrating an example of a functional configuration of a tactile sensor according to a second embodiment. A tactile sensor 12A according to the second embodiment will be described with reference to the same drawing. The tactile sensor 12A is an example of the pressure sensor 12. In the description of the second embodiment, the configurations already described in the first embodiment are denoted by the same reference signs, and descriptions thereof may be omitted.

The tactile sensor 12A includes an imaging unit 123, an image information acquisition unit 124, an image processing unit 125, a tactile information calculation unit 126, and a tactile information output unit 127. The tactile sensor 12A detects tactile information when the contact surface 121 is pressed against a target position to be measured, and outputs the detected information as pressure information IP to the pressure information acquisition unit 120.

The imaging unit 123 captures an image of a degree of deformation of the contact surface 121 when the contact surface 121 is pressed against the target position to be measured. Specifically, the image of the degree of deformation of the contact surface 121 is captured by capturing an image of a marker attached to a non-contact surface 122 that is the back surface of the contact surface 121. The imaging unit 123 may be provided inside the tactile sensor 12A, and may capture an image of the outside of the tactile sensor 12A through the contact surface 121 from the inside of the tactile sensor 12A at the same time as capturing the image of the marker.

The image information acquisition unit 124 acquires information about the image captured by the imaging unit 123. Specifically, the image information acquisition unit 124 acquires the image information captured by the imaging unit 123.

The image processing unit 125 performs image processing on the image information acquired by the image information acquisition unit 124. Specifically, the image processing unit 125 specifies a position of the marker attached to the non-contact surface 122 by the image processing, and calculates a displacement amount of the specified position of the marker.

The tactile information calculation unit 126 calculates tactile information based on the displacement amount of the position of the marker calculated by the image processing unit 125. The tactile information includes information related to a pressure when the contact surface 121 is pressed against the target position to be measured, and also includes information such as a direction in which the pressure is applied. For example, when the measurement target is an eyeball, the tactile information may include information indicating which position of the eyeball having a spherical shape is being pressed.

The tactile information output unit 127 outputs the calculated tactile information as pressure information IP. The pressure information IP may include information about an image captured by the imaging unit 123 in addition to the information related to the pressure.

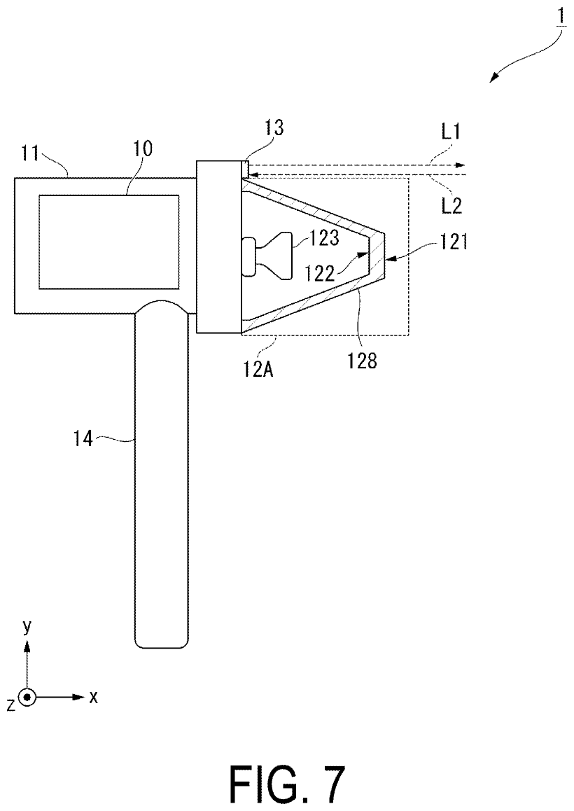

FIG. 7 is a diagram illustrating an example of a functional configuration of a hardness measurement device according to the second embodiment. The functional configuration of the tactile sensor 12A will be described with reference to the same drawing. In the description of the same drawing, a posture of the hardness measurement device 1 may be indicated by using a three-dimensional orthogonal coordinate system of an x-axis, a y-axis, and a z-axis.

The tactile sensor 12A includes the contact surface 121 and the non-contact surface 122 that is the back surface of the contact surface. In addition, the tactile sensor 12A includes a deformation section 128 that deforms in response to a pressure when the contact surface 121 comes into contact with a target position. In other words, a target object side (that is, an outer side) of the deformation section 128 is the contact surface 121, and an imaging unit side (that is, an inner side) of the deformation section 128 is the non-contact surface 122.

The deformation section 128 is made of a soft and transparent material such as silicone. In addition, a shape of the deformation section 128 may be formed according to the shape of a portion to be measured. For example, when the hardness measurement device 1 measures an intraocular pressure, the deformation section 128 may be formed in a shape that fits the shape of an eyelid. In the following description, the deformation section 128 will be also referred to as a probe.

Moreover, the deformation section 128 may be configured to be replaceable. A user may replace the deformation section 128 according to a target to be measured. In addition, the user may periodically replace the deformation section 128 in order to suppress occurrence of a measurement error due to deterioration.

The deformation section 128 includes the non-contact surface 122 marker that is the back side of the contact surface 121.

The tactile sensor 12A includes the imaging unit 123. The imaging unit 123 captures an image of the marker from the back side of the contact surface 121. The imaging unit 123 captures an image of the non-contact surface 122 that is the back surface of the contact surface 121.

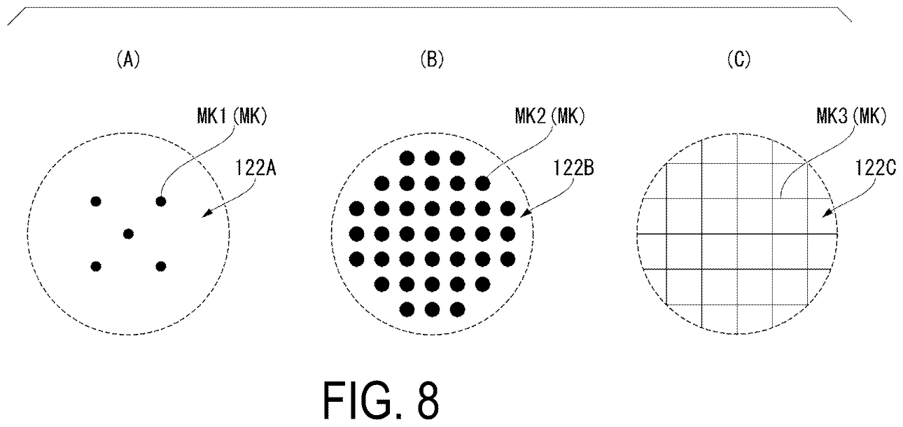

FIG. 8 is a diagram for explaining the marker included in the tactile sensor according to the second embodiment. An example of a marker MK will be described with reference to the same drawing. FIG. 8 illustrates an example of an image of the deformation section 128 captured by the imaging unit 123.

FIG. 8(A) illustrates an example in which five markers MK1 each of which has a dot shape are provided in the deformation section 128, as an example of the marker MK. When the deformation section 128 deforms due to contact of the contact surface 121 with a target position, the sizes of the markers MK1 change. In the description in detail with reference to FIG. 7, the contact surface 121 comes into contact with the target position, which pushes the deformation section 128 in the negative direction of the x-axis. Thus, the markers MK1 approach the deformation section 128. Therefore, the sizes of the markers MK1 become large in an image captured by the imaging unit 123. The tactile information calculation unit 126 may calculate a pressure based on an average value of the sizes of the five markers MK.

In addition, in the example in FIG. 8(A), since the five markers MK1 each of which has a dot shape are provided, positional relationships among the markers MK1 may change depending on the shape or hardness of a target object with which the contact surface 121 comes into contact. The tactile information calculation unit 126 calculates tactile information based on changes in the sizes and the positions of these markers MK1. The tactile information calculation unit 126 may specify the shape of the contacting target object, estimate a position where the contact surface 121 is supposed to be pressed, and perform correction based on the estimated pressed position.

FIG. 8(B) illustrates an example in which a large number of markers MK2 each of which has a dot shape are provided in the deformation section 128, as an example of the marker MK. The number and arrangement of the markers MK are not limited to the positions illustrated in FIG. 8(A), and a large number of markers MK2 may be provided as illustrated in FIG. 8(B). By providing the large number of markers MK2, it is possible to more accurately grasp the shape of the contacting target object.

FIG. 8(C) illustrates an example in which a marker MK3 having a lattice shape is provided in the deformation section 128, as an example of the marker MK. The shape of the marker MK is not limited to the dot shape as illustrated in FIG. 8(A) or FIG. 8(B), and may be, for example, a lattice shape. It is preferable to select the marker MK suitable for the shape and hardness of a target with which the contact surface 121 comes into contact.

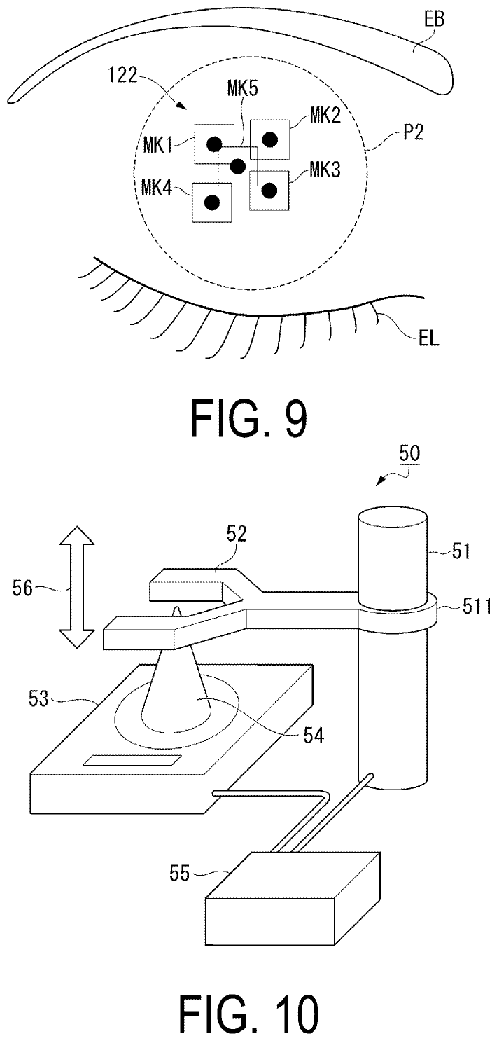

FIG. 9 is a diagram for explaining deformation of a marker when the contact section included in the tactile sensor according to the second embodiment comes into contact with an eyelid. Displacement of the marker MK will be described with reference to the same drawing.

In the example illustrated in the same drawing, a position P2 between an eyebrow EB and an eyelash EL (that is, an eyelid) is set as a target position, and displacement of markers MK provided on the non-contact surface 122 when the contact surface 121 is pressed against the target position will be described. The same drawing illustrates an image of the non-contact surface 122 captured by the imaging unit 123. In the example illustrated in the same drawing, the deformation section 128 includes five markers, namely, a marker MK1, a marker MK2, a marker MK3, a marker MK4, and a marker MK5. The position of each marker MK is displaced because the contact surface 121 is being pressed against the target position. The tactile sensor 12A calculates tactile information based on the displacement.

Note that since the deformation section 128 is formed of a transparent material, the imaging unit 123 may capture an image near the position P2 (for example, opening/closing states of the eyelid, a motion of the eyebrow EB or the eyelash EL, or the like) and perform correction based on the captured image.

### Concept Verification

FIG. 10 is a diagram for explaining a device used for verifying a concept of the hardness measurement device according to the second embodiment. The concept verification of the hardness measurement device 1 will be described with reference to the same drawing. The concept verification of the hardness measurement device 1 was performed by using a verification system 50.

The verification system 50 includes an arm moving device 51, an arm 52, a precision electronic balance 53, a probe 54, and an information processing device 55.

The arm 52 fixes the head of a person. The arm 52 is supported by the arm moving device 51 with an arm support section 511. The arm moving device 51 vertically moves the arm support section 511 in the direction of an arrow 56 to vertically move the head of the person in the direction of the arrow 56. A distance between the head of the person and a top plate (weighing pan) of the precision electronic balance 53 is controlled by the vertical movement of the head of the person in the direction of the arrow 56.

The precision electronic balance 53 includes the probe 54 on the top plate (weighing pan). The probe 54 comes into contact with and is pressed against the eyelid that is a target position of a subject S. By controlling a position of the arm 52, the information processing device 55 controls an amount (a push-in amount) by which the probe 54 is pressed against the eyelid of the subject S. Additionally, the precision electronic balance 53 measures a force when the probe 54 is pressed against the eyelid of the subject S, and outputs the force to the information processing device 55. The information processing device 55 stores a correspondence relationship between the push-in amount and the obtained force.

The information processing device may be, for example, a personal computer or the like.

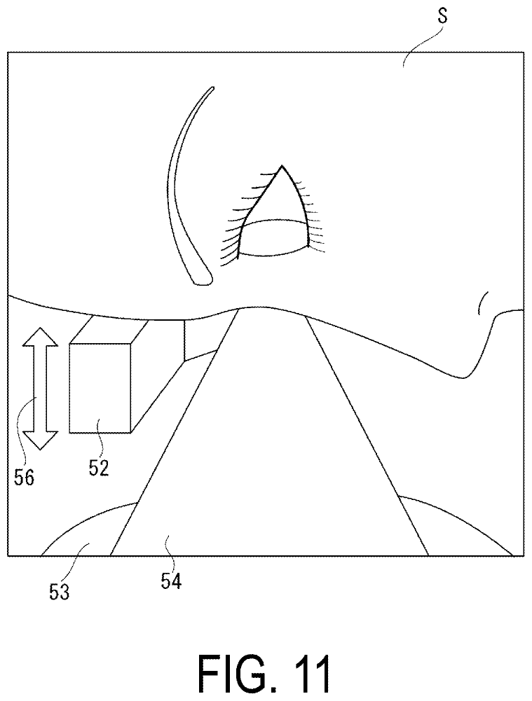

FIG. 11 is a diagram for explaining a method used for verifying the concept of the hardness measurement device according to the second embodiment. The method used for verifying the concept will be described with reference to the same drawing. In the same drawing, the subject S fixes his/her head to the arm 52, and pushes the eyelid of the right eye against the probe 54. The arm 52 moves up and down in the direction of the arrow 56 to change the push-in amount, and the precision electronic balance 53 measures a force corresponding to the push-in amount. The information processing device 55 stores a correspondence relationship between the push-in amount and the obtained force.

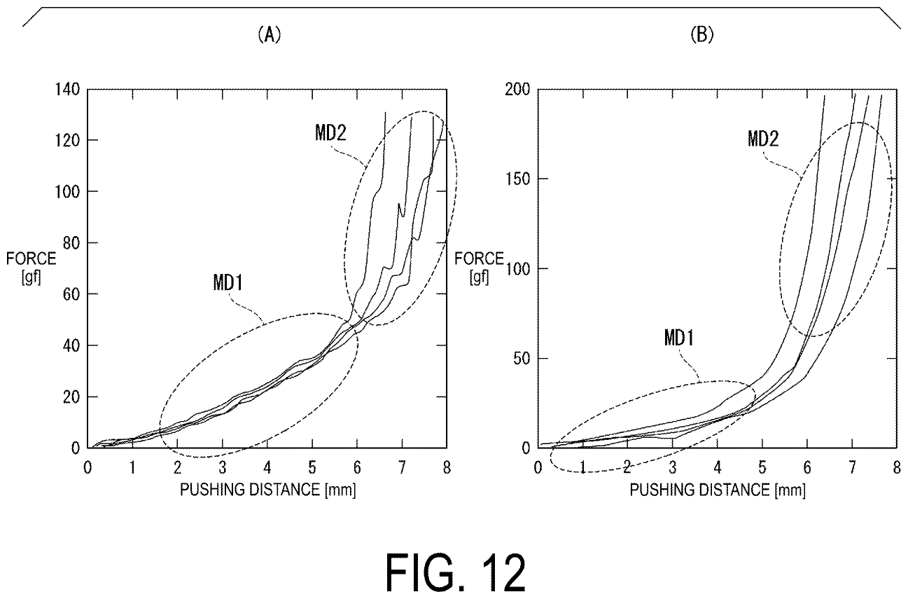

FIG. 12 is a diagram for explaining an example of measurement results measured by the concept verification of the hardness measurement device according to the second embodiment. An example of the measurement results measured by the concept verification will be described with reference to the same drawing. In the same drawing, measurement results of two different subjects S are illustrated. In the same drawing, the horizontal axis represents a push-in amount and the vertical axis represents a force, and the correspondence relationship thereof is illustrated. FIG. 12(A) illustrates measurement results of a subject S1, and FIG. 12(B) illustrates measurement results of a subject S2.

First, the measurement results of the subject S1 will be described with reference to FIG. 12(A). As illustrated in the same drawing, it was confirmed that the measurement results tend to have two different inclinations depending on the push-in amount. To be specific, it was confirmed that the inclination of a mode MD1 was present in a range of the push-in amount from 0 mm (millimeters) to 6 mm, and the inclination of a mode MD2 was present in a range of the push-in amount from 6 mm to 8 mm. It is presumed that the mode MD1 is a result obtained from an elastic modulus of an eyelid, and the mode MD2 is a result obtained from an elastic modulus of an eyeball.

That is, in the present concept verification, a first elastic modulus was obtained according to a first push-in amount, and a second elastic modulus was obtained according to a second push-in amount. The first elastic modulus is the elastic modulus of the eyelid, and the second elastic modulus is the elastic modulus of the eyeball. That is, the first elastic modulus is an elastic modulus of the skin or the like of an animal, and the second elastic modulus is an elastic modulus of a tissue existing inside the skin or the like of an animal. Since the inclination of the mode MD2 is steeper than the inclination of the mode MD1, it can be seen that the eyeball is harder than the eyelid.

Next, the measurement results of the subject S2 will be described with reference to FIG. 12(B). It was confirmed that the inclination of the mode MD1 was present in the range of the push-in amount from 0 [mm] to 5.5 [mm], and the inclination of the mode MD2 was present in the range of the push-in amount from 5.5 [mm] to 8 [mm]. Similar to the case of the subject S1, for the subject S2, the result that the inclination of the mode MD2 was steeper than the inclination of the mode MD1 was obtained.

According to the present concept verification, the hardness measurement device 1 can measure the inclination of the mode MD1 by pressing the probe against the target position, and then can measure the inclination of the mode MD2 by pressing the probe more strongly against the target position. That is, it was found that the hardness of a tissue existing inside the skin or the like of an animal can be measured on the skin or the like of the animal.

When the results of the concept verification are applied to the present embodiment, the calculation unit 130 included in the hardness calculation device 10 calculates a first hardness when a distance to a reference position is a first distance, and a second hardness when a distance to the reference position is a second distance. The calculation unit 130 calculates the hardness of a tissue inside the skin of an animal by calculating the first hardness and the second hardness.

In the present embodiment, a part of the body of an animal is, for example, the eyelid of a human. When the hardness measurement device 1 measures the eyelid of a human, the first hardness is a hardness of the eyelid, and the second hardness is a hardness of the eyeball. The first hardness and the second hardness are different from each other.

### Summary of Second Embodiment

According to the embodiment described above, the hardness measurement device 1 measures the first hardness and the second hardness that are different from each other. Thus, the hardness measurement device 1 can measure a part of a tissue inside a body covered with skin. In addition, according to the present embodiment, since a part of a tissue in a body covered with skin can be measured on the skin, measurement can be performed by a method that does not give discomfort to a patient and does not impose a burden on the patient.

Moreover, according to the embodiment described above, since the first hardness is the hardness of an eyelid and the second hardness is the hardness of the eyeball, an intraocular pressure can be measured on the eyelid.

Here, examples of tonometers using conventional techniques include pneumatic tonometers and Goldmann direct type tonometers. There are problems in that a pneumatic tonometer, which is a device that measures an intraocular pressure from the distortion of an eyeball when air is applied to the eyeball, is difficult to move because of its large size, and is expensive. The Goldmann direct type tonometer is a device that ejects a small probe toward an eyeball after application of an eye drop to measure the reaction. Portable Goldmann direct tonometers also exist, but in practice, there are problems in that such tonometers require skill to be used and replacing the probe every time requires time and labor. According to the present embodiment, the hardness measurement device 1 measures the intraocular pressure and hardness of an eyeball itself (dura mater) by pressing the probe not against the eyeball but on the eyelid while gradually applying a force, and measuring a change in the reaction force. Further, according to the present embodiment, since the tactile sensor 12A is used as the pressure sensor 12, the measurement method is easy, and it is possible to provide a small and inexpensive tonometer.

Additionally, according to the embodiment described above, the hardness measurement device 1 includes the tactile sensor 12A as the pressure sensor 12. The tactile sensor 12A includes the deformation section 128 that deforms in response to a pressure applied when the contact surface 121 comes into contact with a target position, the marker MK included in the deformation section 128, and the imaging unit 123 that captures an image of the marker MK, thereby detecting a tactile sense when coming into contact with the target position. According to the present embodiment, the tactile sensor 12A can further reduce the size of the entire device by using a small camera as the imaging unit 123.

In addition, the tactile sensor 12A includes a plurality of markers MK, and thus, can measure triaxial force information at multiple points (a plurality of positions).

In addition, the tactile sensor 12A includes the deformation section 128 formed into a freely selected shape, which allows the shape of the probe according to the shape of an eyeball, an eyelid, or other portions of a body to be measured to be easily designed. By using the probe matched with the shape of a target to be measured, the hardness measurement device 1 can measure the hardness of the target to be measured with higher accuracy. In addition, since the hardness measurement device 1 can use the probe corresponding to the shape of the target to be measured, which allows measurement to be safely performed without imposing a burden on the patient.

Additionally, according to the above-described embodiment, since the hardness measurement device 1 includes the tactile sensor 12A as the pressure sensor 12, the hardness measurement device 1 itself can be made inexpensive. In addition, the hardness measurement device 1 can be made further inexpensive by using the probe as a consumable. In addition, since the probe is a consumable, even when a plurality of users share one hardness measurement device 1, there is no concern about infection or the like, and the hardness measurement device 1 can be safely used.

Additionally, according to the above-described embodiment, since the hardness measurement device 1 includes the tactile sensor 12A as the pressure sensor 12, the probe can be easily attached and detached. Since the probe can be easily attached to and detached from the hardness measurement device 1, a user can detach the probe once, clean and disinfect the probe with alcohol cotton or the like, and then attach the probe to the hardness measurement device 1 again, thereby allowing the probe to be repeatedly used.

### Third Embodiment

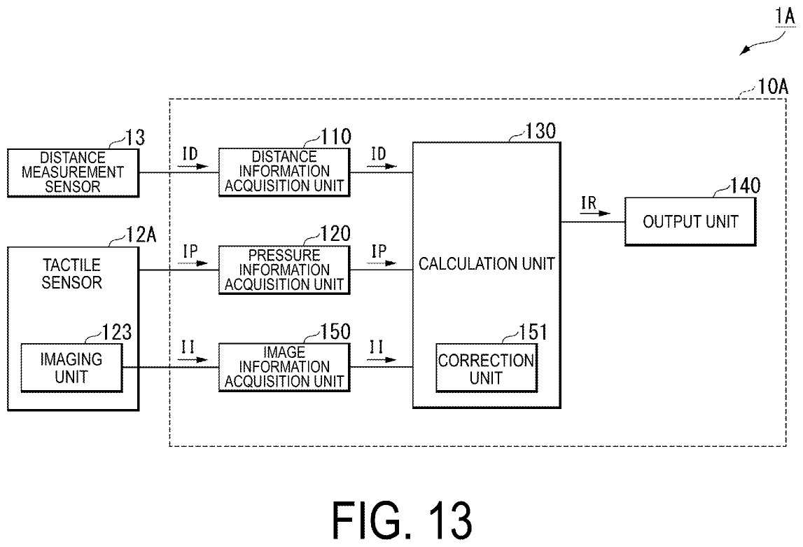

FIG. 13 is a diagram illustrating an example of a functional configuration of a hardness calculation device according to a third embodiment. A hardness calculation device 10A according to the third embodiment will be described with reference to the same drawing. In the description of the hardness calculation device 10A, configurations similar to those of the hardness calculation device 10 are denoted by the same reference signs, and descriptions thereof may be omitted. The hardness calculation device 10A is different from the hardness calculation device 10 in that the hardness calculation device 10A includes an image information acquisition unit 150 and a correction unit 151.

The hardness measurement device 1A according to the third embodiment includes the tactile sensor 12A as the pressure sensor 12. The tactile sensor 12A includes the imaging unit 123. The deformation section 128 included in the tactile sensor 12A is made of a transparent material, and the imaging unit 123 captures an image of both the marker MK and an object existing on the contact surface 121 side of the deformation section 128. The imaging unit 123 outputs the captured image to the image information acquisition unit 150 as image information II.

The image information acquisition unit 150 acquires the image information II from the tactile sensor 12A. The image information acquisition unit 150 provides the acquired image information II to the correction unit 151. The calculation unit 130 corrects hardness information IR based on the acquired image information II.

For example, the image information II includes an image in which the state of an eyelid is captured. The correction unit 151 corrects the hardness information IR according to the state of the eyelid specified by using the image information II. The state of the eyelid means, for example, information indicating whether the eyelid is in a strongly closed state or in a half-opened state, or the like.

The output unit 140 may output both the hardness information IR calculated by the calculation unit 130 and the image information II including information about the captured image.

### Summary of Third Embodiment

As described above, according to the present embodiment, a hardness measurement device 1A acquires information of an image captured by the imaging unit 123. That is, according to the present embodiment, the tactile sensor 12A acquires image information about a target position in addition to information about a pressure. Thus, according to the present embodiment, more information can be acquired, and correction based on the acquired image information can be performed.

### Fourth Embodiment

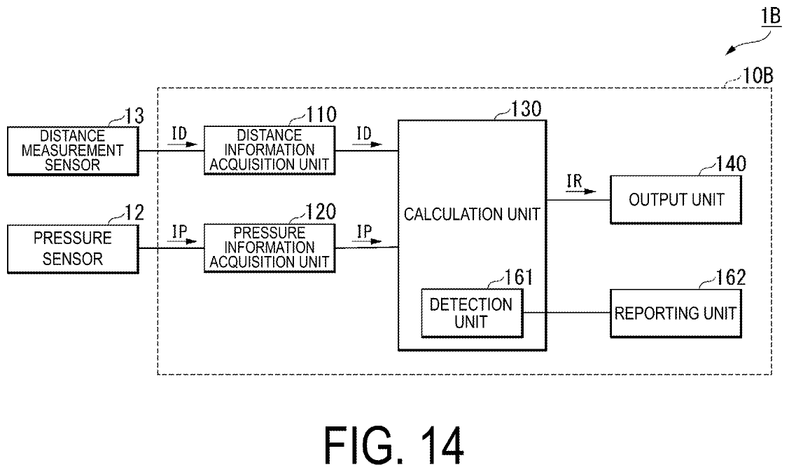

FIG. 14 is a diagram illustrating an example of a functional configuration of a hardness calculation device according to a fourth embodiment. A hardness calculation device 10B according to the fourth embodiment will be described with reference to the same drawing. In the description of the hardness calculation device 10B, configurations similar to those of the hardness calculation device 10 are denoted by the same reference signs, and descriptions thereof may be omitted. The hardness calculation device 10B is different from the hardness calculation device 10 in that the hardness calculation device 10B includes a detection unit 161 and a reporting unit 162.

The detection unit 161 detects the fact that measurement by the hardness calculation device 10B has ended. For example, the detection unit 161 distinctively specifies the first hardness and the second hardness from a hardness calculated by the calculation unit 130, and notifies the reporting unit 162 when measurement of the second hardness is finished.

When receiving the notification from the detection unit 161, the reporting unit 162 reports the fact that the measurement has ended. The reporting unit 162 includes, for example, a buzzer, and reports the end of the measurement by using a buzzer sound.

It is to be noted that 162 may report the end of the measurement by other means such as vibration or the like.

### Summary of Fourth Embodiment

As described above, according to the present embodiment, the hardness calculation device 10B reports the end of the measurement to the user. To be more specific, the hardness calculation device 10B includes the detection unit 161 to distinctively specify the first hardness and the second hardness, and notifies the reporting unit 162 when the measurement of the second hardness is finished. Since the hardness calculation device 10B includes the reporting unit 162, when the reporting unit 162 receives the notification, the hardness calculation device 10B notifies the user by using a buzzer sound or the like. Thus, according to the present embodiment, the user who uses the hardness calculation device 10B can recognize the end of the measurement, and can recognize how far the probe needs to be pushed into the target position. Thus, according to the present embodiment, it is possible to prevent the measurement from being finished before the measurement of the second hardness, and it is possible to prevent the probe or the affected area that is the target position from being damaged due to excessive push-in of the probe by the user. Further, according to the present embodiment, even a general user who is not a skilled doctor can easily and safely perform measurement by using the hardness calculation device 10B.

### Fifth Embodiment

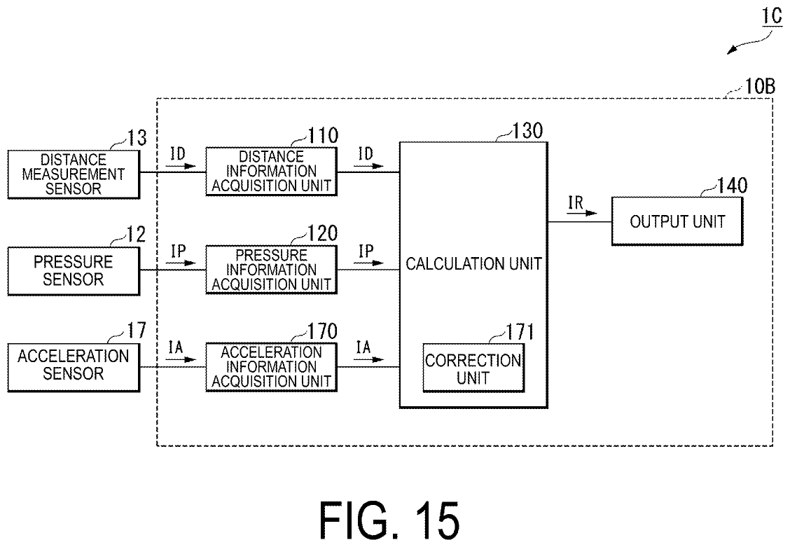

FIG. 15 is a diagram illustrating an example of a functional configuration of a hardness calculation device according to a fifth embodiment. A hardness calculation device 10C according to the fifth embodiment will be described with reference to the same drawing. In the description of the hardness calculation device 10C, configurations similar to those of the hardness calculation device 10 are denoted by the same reference signs, and descriptions thereof may be omitted. The hardness calculation device 10C is different from the hardness calculation device 10 in that the hardness calculation device 10C includes an acceleration information acquisition unit 170 and a correction unit 171. Additionally, the hardness measurement device 1C according to the present embodiment is different from the hardness measurement device 1 in that the hardness measurement device 1C further includes an acceleration sensor 17.

The acceleration sensor 17 detects triaxial acceleration. The acceleration sensor 17 detects an inclination of the hardness measurement device 1C by detecting triaxial acceleration. The acceleration sensor 17 is also referred to as a posture sensor. The acceleration sensor 17 outputs the detected inclination as acceleration information IA to the acceleration information acquisition unit 170.

The acceleration information acquisition unit 170 acquires the acceleration information IA from the acceleration sensor 17. The acceleration information acquisition unit 170 outputs the acquired acceleration information IA to the calculation unit 130.

The calculation unit 130 includes the correction unit 171. The correction unit 171 corrects hardness information IR according to the acquired acceleration information IA. That is, the correction unit 171 corrects the calculated hardness indicated by the hardness information IR according to the information indicating the inclination.

The output unit 140 outputs both the hardness information IR and the information indicating the inclination detected when the hardness information IR is calculated.

Note that the hardness calculation device 10C may store the hardness information IR and a posture when the hardness information IR is measured in association with each other. Since the hardness calculation device 10C has information of the hardness information IR for each posture, the hardness calculation device 10C can perform correction according to the posture.

### Summary of Fifth Embodiment

As described above, the hardness measurement device 1C further includes the acceleration sensor 17 and detects an inclination of the hardness measurement device 1C. In addition, the hardness measurement device 1C outputs the hardness information IR and the information indicating the inclination detected when the hardness information IR is calculated in association with each other. Thus, according to the hardness measurement device 1C, a difference in measurement value due to a difference in posture can be measured.

In addition, the hardness measurement device 1C includes the correction unit 171, thereby performing correction according to the posture. Thus, according to the hardness measurement device 1C, the difference in measurement value due to the difference in posture can be corrected. Note that the difference in posture may be, for example, a difference in a lying state, a sitting state, or the like.

Further, in the above-described embodiment, when the tactile sensor 12A is used as the pressure sensor 12, the device itself is not affected by a gravitational acceleration, so that the measurement can be performed in any posture.

### Sixth Embodiment

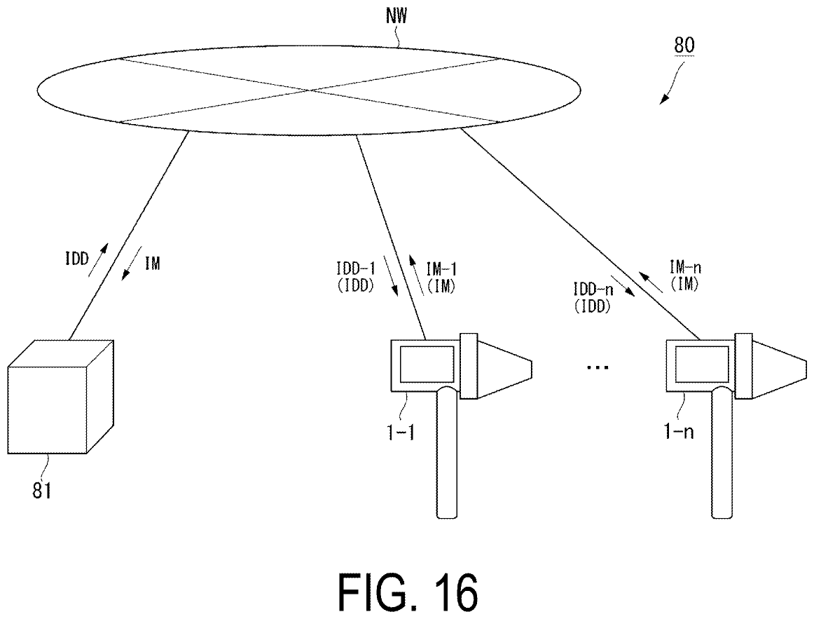

FIG. 16 is a diagram illustrating an example of a diagnostic system according to a sixth embodiment. A diagnostic system 80 will be described with reference to the same drawing. The diagnostic system 80 includes a diagnostic device 81 and a plurality of hardness measurement devices 1. In the same drawing, as an example of the plurality of hardness measurement devices 1, a hardness measurement device 1-1, . . . , a hardness measurement device 1-n (n is a natural number) are illustrated.

The hardness measurement device 1 is held by each user. The hardness measurement device 1 measures an intraocular pressure of a user and outputs measurement information IM that is a measurement result through a predetermined network NW. For example, the hardness measurement device 1-1 outputs measurement information IM-1, and the hardness measurement device 1-n outputs measurement information IM-n.

The diagnostic device 81 acquires measurement information IM from each of the plurality of hardness measurement devices 1. The diagnostic device 81 performs diagnosis according to the intraocular pressure of the user holding the hardness measurement device 1 based on the acquired measurement information IM. For example, the diagnostic device 81 may perform diagnosis by a doctor checking the measurement information IM, or may perform diagnosis based on statistical information stored in advance. The diagnostic device 81 outputs diagnostic information IDD that is a result of the diagnosis. Note that the diagnostic information IDD may include, in addition to a disease name and a medical condition that have been obtained as a result of diagnosis, information about a required drug or information about a treatment required for the user.

The hardness measurement device 1 acquires the diagnostic information IDD from the diagnostic device 81 through the predetermined network NW. For example, the hardness measurement device 1-1 acquires diagnostic information IDD-1, and the hardness measurement device 1-n acquires diagnostic information IDD-n. The hardness measurement device 1 performs an operation according to the acquired diagnostic information IDD. For example, in a case where the diagnostic information IDD includes a disease name and a medical condition that have been obtained as a result of diagnosis, information about a necessary drug, and the like, the result is output to a display unit (not illustrated) or the like. When the diagnostic information IDD includes a treatment required for the user, the hardness measurement device 1 performs the treatment according to the information included in the diagnostic information IDD.

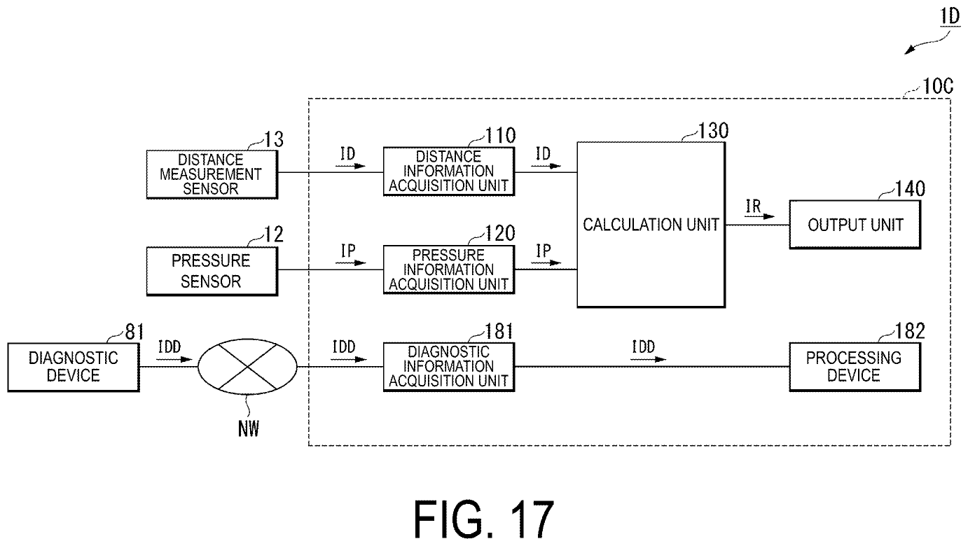

FIG. 17 is a diagram illustrating an example of a functional configuration of the hardness calculation device according to the sixth embodiment. A hardness calculation device 10D according to the sixth embodiment will be described with reference to the same drawing. In the description of the hardness calculation device 10D, configurations similar to those of the hardness calculation device 10 are denoted by the same reference signs, and descriptions thereof may be omitted. The hardness calculation device 10D is different from the hardness calculation device 10 in that the hardness calculation device 10D includes a diagnostic information acquisition unit 181 and a treatment device 182.

The diagnostic information acquisition unit 181 acquires diagnostic information IDD from the diagnostic device 81 through the predetermined network NW. The diagnostic information IDD is information including a result diagnosed by the diagnostic device 81 according to a result of the hardness information IR output by the output unit 140. That is, the diagnostic information acquisition unit 181 acquires the diagnostic information IDD that is information acquired according to the result of the hardness information IR output by the output unit 140.

The treatment device 182 performs a predetermined treatment included in the diagnostic information IDD acquired by the diagnostic information acquisition unit 181. Here, examples of the predetermined treatment widely include medical care, treatment, and the like to be performed by giving stimulation such as massage, an electric signal, or wind pressure to the user. For example, when the treatment device 182 performs massage, the hardness measurement device 1D includes a drive unit (not illustrated), and performs massage of an eyelid or the like by driving the drive unit. The diagnostic information IDD may include information such as a force and time to be required for massage and a position where the massage is performed. The hardness measurement device 1 may prompt the user to press the treatment device 182 against a predetermined position by using voice.

### Summary of Sixth Embodiment

As described above, according to the present embodiment, the hardness measurement device ID includes the diagnostic information acquisition unit 181 to acquire diagnostic information IDD, and includes the treatment device 182 to perform a predetermined treatment indicated in the diagnostic information IDD. Thus, according to the present embodiment, it is possible to perform a treatment according to a result measured by the hardness measurement device 1. Accordingly, the user can take an inspection without regularly going to see a doctor and can easily receive an appropriate treatment.

Further, according to the present embodiment, since the diagnostic device 81 can acquire hardness information IR and the hardness measurement device ID can acquire diagnostic information IDD, a doctor can see measurement results by a plurality of users through the diagnostic device 81 and can give instructions to the plurality of users in response to the measurement results. Thus, the doctor can remotely obtain the information of operations and can give instructions according to the obtained information. That is, according to the present embodiment, remote medical care can be easily achieved.

### OTHER EMBODIMENTS

In the embodiments described above, an example in which the hardness measurement device 1 is for medical use has been described. However, the hardness measurement device 1 according to the present embodiment is not limited to medical use, and may be used for cosmetic use, for example. When the hardness measurement device 1 is used for cosmetic use, the hardness measurement device 1 may measure or estimate a tension of the skin, a fat amount, a muscle amount, or the like. Furthermore, the hardness measurement device 1A according to the present embodiment can perform more advanced measurement by capturing an image of a skin condition at the same time as measuring the tension of the skin, the fat amount, or the like. Furthermore, the hardness measurement device ID may perform massage, electrical stimulation, or the like according to the measured skin condition or the like.

In the embodiments described above, an example of a case where the hardness measurement device 1 measures the hardness of a part of the body of an animal has been described. However, the hardness measurement device 1 may measure the hardness of an inspection target object such as food, materials, or other objects. For example, when the hardness measurement device 1 detects the hardness of a steamed bun with a bean-jam filling (manju) as an example of food, the hardness measurement device 1 can measure the hardness of the skin as the first hardness and measure the hardness of the bean-jam filling inside the skin as the second hardness. For example, when the hardness measurement device 1 detects the hardness of an elastic member as an example of a material, a degree of deterioration or the like can be detected by measuring the hardness of the outside of the elastic member and the hardness of the inside of the elastic member while distinguishing the hardness of the outside of the elastic member and the hardness of the inside of the elastic member from each other.

For example, when the inspection target object is a soft object, it is possible to measure the hardness of the inspection target object that is the soft object by using a soft substance as the material of the deformation section 128 according to the hardness of the inspection target object.

Note that all or some of the functions of the units and sections that are included in the hardness measurement device 1 in the above-described embodiments may be achieved by recording a program for achieving these functions in a computer-readable recording medium and causing a computer system to read and execute the program recorded in the recording medium. Note that the “computer system” here includes an OS and hardware such as peripheral devices.

Further, the “computer-readable recording medium” refers to a portable medium such as a magneto-optical disk, a ROM, or a CD-ROM, or a storage unit such as a hard disk built in the computer system. Furthermore, the “computer-readable recording medium” may include a medium that dynamically holds a program for a short period of time, such as a communication line in a case of transmitting a program through a network such as the Internet, and a medium that holds a program for a certain period of time, such as a volatile memory inside the computer system serving as a server or a client in that case. Further, the program described above may be a program for achieving some of the above-described functions, or may be a program capable of achieving the above-described functions in combination with a program already recorded in the computer system.

Although the formation for exploiting the present invention has been described above by using the embodiments, the present invention is not limited to these embodiments at all, and various modifications and substitutions can be made without departing from the scope of the present invention.

### REFERENCE SIGNS LIST

1 Hardness measurement device, 10 Hardness calculation device, 11 Housing section, 12 Pressure sensor, 12A Tactile sensor, 13 Distance measurement sensor, 14 Hold section, 17 Acceleration sensor, 110 Distance information acquisition unit, 120 Pressure information acquisition unit, 130 Calculation unit, 140 Output unit, 150 Image information acquisition unit, 151 Correction unit, 161 Detection unit, 162 Reporting unit, 170 Acceleration information acquisition unit, 171 Correction unit, 181 Diagnostic information acquisition unit, 182 Treatment device, 121 Contact surface, 122 Non-contact surface, 123 Imaging unit, 124 Image information acquisition unit, 125 Image processing unit, 126 Tactile information calculation unit, 127 Tactile information output unit, 50 Verification system, 51 Arm moving device, 511 Arm support section, 52 Arm, 53 Precision electronic balance, 54 Probe, 55 Information processing device, 56 Arrow, 80 Diagnostic system, 81 Diagnostic device, S Subject, ID Distance information, IP Pressure information, II Image information, IM Measurement information, LA Acceleration information, IDD Diagnostic information

1. A hardness calculation device comprising:
a distance measurement information acquisition unit configured to acquire distance measurement information indicating a distance to a reference position that is a part of a body of an animal;
a pressure information acquisition unit configured to acquire pressure information indicating a pressure when a contact section is pressed against a target position that is a part of the body of the animal, the target position being a position different from the reference position;
a calculation unit configured to calculate hardness information that is information about a hardness of a tissue existing in the body of the animal at the target position based on the distance measurement information acquired by the distance measurement information acquisition unit and the pressure information acquired by the pressure information acquisition unit; and
an output unit configured to output the hardness information calculated by the calculation unit.

2. The hardness calculation device according to claim 1,
wherein the distance measurement information acquisition unit acquires a plurality of pieces of the distance measurement information acquired at different moments from each other,
the pressure information acquisition unit acquires a plurality of pieces of the pressure information acquired at moments corresponding to the moments at which the plurality of pieces of the distance measurement information are acquired, and
the calculation unit calculates the hardness information based on the acquired plurality of pieces of distance measurement information and the acquired plurality of pieces of pressure information.

3. The hardness calculation device according to claim 2,
wherein the calculation unit calculates a first hardness when a distance to the reference position is a first distance and a second hardness when a distance to the reference position is a second distance, the second hardness being different from the first hardness.

4. The hardness calculation device according to claim 3,
wherein the part of the body of the animal is an eyelid of a human,
the first hardness is a hardness of the eyelid, and
the second hardness is a hardness of an eyeball.

5. A hardness measurement device comprising:
the hardness calculation device according to claim 1;
a distance measurement sensor configured to measure a distance to the reference position, the distance measurement sensor being configured to output the measured distance to the distance measurement information acquisition unit as the distance measurement information; and
a pressure sensor configured to measure a pressure when the contact section is pressed against the target position, the pressure sensor being configured to output the measured pressure to the pressure information acquisition unit as the pressure information.

6. The hardness measurement device according to claim 5,
wherein the pressure sensor includes
a deformation section including a contact surface including the contact section, the deformation section being configured to deform in response to pressure when the contact surface comes into contact with the target position,
a marker provided on a back side of the contact surface, and
an imaging unit configured to capture an image of the marker from the back side of the contact surface.

7. The hardness measurement device according to claim 6,
wherein the deformation section is made of a transparent material,
the imaging unit captures an image of both the marker and an object existing on a contact surface side of the deformation section, and
the output unit outputs both the hardness information and the captured image.

8. The hardness measurement device according to claim 5,
wherein the distance measurement sensor measures the distance to the reference position without contact.

9. The hardness measurement device according to claim 5, further comprising:
a posture sensor configured to detect at least an inclination,
wherein the output unit outputs both the hardness information and information indicating the inclination detected when the hardness information is calculated.

10. The hardness measurement device according to claim 9,
wherein the calculation unit further includes a correction unit configured to correct the hardness indicated by the calculated hardness information according to the information indicating the inclination.

11. The hardness measurement device according to claim 5, further comprising:
a diagnostic information acquisition unit configured to acquire diagnostic information that is information to be acquired according to a result of the hardness information output by the output unit; and
a treatment device configured to perform a treatment included in the diagnostic information.

12. A hardness calculation method comprising:
acquiring distance measurement information indicating a distance to a reference position that is a part of a body of an animal;
contacting a target position that is a part of the body of the animal, the target position being a position different from the reference position;
acquiring pressure information indicating a pressure applied when pressing against the target position by the contacting of the target position;
calculating hardness information that is information about a hardness of a tissue existing in the body of the animal at the target position based on the distance measurement information acquired by the acquiring of distance measurement information, and the pressure information acquired by the acquiring pressure information; and
outputting the hardness information calculated in the calculating hardness information.

13. A hardness calculation device comprising:
a distance measurement information acquisition unit configured to acquire distance measurement information indicating a distance to a reference position of an inspection target object;
a pressure information acquisition unit configured to acquire pressure information indicating a pressure when a contact section is pressed against a target position that is a part of the inspection target object, the target position being a position different from the reference position;
a calculation unit configured to calculate hardness information that is information about a hardness of a tissue existing in a body of the animal at the target position based on the distance measurement information acquired by the distance measurement information acquisition unit and the pressure information acquired by the pressure information acquisition unit; and
an output unit configured to output the hardness information calculated by the calculation unit.
